(19) United States
(12) Patent Application Publication
(10) Pub. No.: US 2026/0366442 A1
(43) Pub. Date: Nov. 19, 2026

(54) METHOD AND MODEL GENERATING SYSTEM FOR GENERATING AN OIL QUALITY MONITORING MODEL, AND METHOD AND DETECTION SYSTEM FOR DETECTING AN ANOMALY IN AN OIL PRESSURE RELATED OPERATIONAL CONDITION

(71) Applicant: Intangles Lab Private Ltd., Pune (IN)

(72) Inventor: Abhijit Vishwas PATIL, Pune (IN)

(21) Appl. No.: 19/211,984

(22) Filed: May 19, 2025

(51) Int. Cl. F01M 11/10; G01N 33/28; G07C 5/08; G07C 5/10

(52) U.S. Cl. CPC F01M 11/10; G01N 33/2888; G07C 5/085; G07C 5/10; F01M 2011/14; F01M 2011/1446; F01M 2011/1473

(57) ABSTRACT

A method for generating an oil quality monitoring model for an internal combustion engine of a vehicle. The method comprises receiving, from an engine management system of the internal combustion engine via a data interface, engine model data obtained periodically during a sequence of sampling time periods during operation of the internal combustion engine. The engine model data comprises oil pressure data indicative of oil pressure of the internal combustion engine, and engine speed data indicative of an engine speed of the engine. The method includes storing the engine model data in a memory for each sampling time period and dividing, by a processor, the oil pressure data into a predetermined number of engine speed bins derived from the engine speed data during each sampling time period so as to generate binned engine model data indicative of frequency of occurrence of oil pressures within each bin for each sampling time period. The method further includes generating, by the processor for each bin of the predetermined number of bins and for each sampling time period, statistical attribute model data based on the binned engine model data, and generating, by the processor, the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions. The method additionally includes storing, in the memory, the oil quality monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

### TECHNICAL FIELD

The present disclosure relates to a method and model generating system for generating an oil quality monitoring model for an internal combustion engine, and a method and detection system for detecting an anomaly in an oil pressure related operational condition. Examples of the disclosure relate as well to computer programs for executing the methods and non-transitory tangible computer-readable media having stored thereon a computer program for executing the methods.

### BACKGROUND

Internal combustion engines typically use lubricating oils for a variety of purposes. For example, lubricating oils may be used to help reduce wear of components such as bearings, pistons, piston rings, cylinder liners and the valve train, help reduce friction of boundary and hydrodynamically lubricated components, for piston cooling, for corrosion prevention due to acids and moisture, for cleaning pistons and preventing sludge build-up on internal surfaces, and for keeping seals lubricated and controlling swelling to prevent leakage due to seal failure.

Generally, lubricating oil for an internal combustion engine should be maintained at optimal pressure to help ensure it reaches all parts of the engine, provides consistent lubrication, and minimise wear and tear. Furthermore, optimal oil pressure helps ensure that oil circulates effectively, carrying away heat and helping to maintain the engine at the optimal temperature, for example, where it may operate most efficiently. Additionally, the optimal pressure of lubricating oil helps ensure that the oil flows through the engine and the filter, so helping to keep the engine clean.

However, deviations of oil pressure from an optimal range, for example, may lead to engine inefficiencies, increased sludge build up, leaks, reduced engine lifetime or reduced service intervals. Sometimes, it may not be apparent that there is a problem with the oil and oil pressure until one or more faults of the engine actually occur, for example generating a fault code. This may lead to increased down time while the vehicle is repaired, increasing costs, or may cause damage to the engine and its parts, as well as other problems with engine operation.

It is an object of the subject matter of the present disclosure to at least address or alleviate the above problems.

### SUMMARY

Examples of the present disclosure seek to address or at least alleviate the above problems.

In a first aspect, there is provided a method for generating an oil quality monitoring model for an internal combustion engine of a vehicle, the method comprising: receiving, from an engine management system of the internal combustion engine via a data interface, engine model data obtained periodically during a sequence of sampling time periods during operation of the internal combustion engine, the engine model data comprising oil pressure data indicative of oil pressure of the internal combustion engine, and engine speed data indicative of an engine speed of the engine; storing the engine model data in a memory for each sampling time period; dividing, by a processor, the oil pressure data into a predetermined number of engine speed bins derived from the engine speed data during each sampling time period so as to generate binned engine model data indicative of frequency of occurrence of oil pressures within each bin for each sampling time period; generating, by the processor for each bin of the predetermined number of bins and for each sampling time period, statistical attribute model data based on the binned engine model data; generating, by the processor, the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions; and storing, in the memory, the oil quality monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

In a second aspect, there is provided a model generating system for generating an oil quality monitoring model for an internal combustion engine of a vehicle, the system comprising: a data interface configured to receive, from an engine management system of the internal combustion engine via a data interface, engine model data obtained periodically during a sequence of sampling time periods during operation of the internal combustion engine, the engine model data comprising oil pressure data indicative of oil pressure of the internal combustion engine, and engine speed data indicative of an engine speed of the engine; a memory configured to store the engine model data for each sampling time period; and a processor configured to: divide the oil pressure data into a predetermined number of engine speed bins derived from the engine speed data during each sampling time period so as to generate binned engine model data indicative of frequency of occurrence of oil pressures within each bin for each sampling time period; generate, for each bin of the predetermined number of bins and for each sampling time period, statistical attribute model data based on the binned engine model data; and generate the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions; in which the memory is configured to store the oil monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

Accordingly, for example, an oil quality monitoring model may be generated that may more accurately take into account measured aspects of oil quality and oil pressure for a wide range of operational conditions of the engine. This may further help improve pre-emptive prediction of possible defects related to oil quality or pressure before an error fault code is generated by the engine control unit (ECU) of the engine.

In a third aspect, there is provided a method for detecting an anomaly in an oil pressure related operational condition of an internal combustion engine based on comparison with an oil quality monitoring model generated according to the first aspect, the method comprising: receiving, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine; storing the engine test data in a memory for each test sampling period; dividing, by a processor, the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model; generating, by the processor for each bin of the predetermined number of bins, statistical attribute test data based on the binned engine test data; comparing, by the processor for each bin, the statistical attribute test data with the statistical attribute model data of the oil quality monitoring model; and generating, by the processor, an alert based on if the comparison between the statistical attribute test data and the statistical attribute model data of the oil quality monitoring model for each test sampling time period meets a predetermined condition.

In a fourth aspect, there is provided a detection system for detecting an anomaly in an oil pressure related operational condition of an internal combustion engine based on comparison with an oil quality monitoring model generated according to the second aspect, the system comprising: a data interface configured to receive, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine; a memory configured to store the engine test data for each test sampling period; and a processor configured to: divide the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model; generate, for each bin of the predetermined number of bins, statistical attribute test data based on the binned engine test data; compare, for each bin, the statistical attribute test data with the statistical attribute model data of the oil quality monitoring model; and generate an alert based on if the comparison between the statistical attribute test data and the statistical attribute model data of the oil quality monitoring model for each test sampling time period meets a predetermined condition.

In a fifth aspect, there is provided a method for detecting an anomaly in an oil pressure related operational condition of an internal combustion engine based on comparison with an oil quality monitoring model, the oil quality monitoring model comprising statistical attribute model data previously generated from binned engine model data of the internal combustion engine, in which the binned engine model data has been generated by dividing oil pressure data of engine model data obtained from the internal combustion engine into a predetermined number of engine speed bins, the engine model data comprises oil pressure model data indicative of a oil pressure of the engine and engine speed model data indicative of an engine speed of the engine, the predetermined number of engine speed bins are derived from the engine speed data during a sampling time period, and the binned engine model data is indicative of frequency of occurrence of oil pressures within each bin, the method comprising: receiving, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine; storing the engine test data in a memory for each test sampling period; dividing, by a processor, the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model; generating, by the processor for each bin of the predetermined number of bins, statistical attribute test data based on the binned engine test data; comparing, by the processor for each bin, the statistical attribute test data with the statistical attribute model data of the oil quality monitoring model; and generating, by the processor, an alert based on if the comparison between the statistical attribute test data and the statistical attribute model data of the oil quality monitoring model for each test sampling time period meets a predetermined condition.

In a sixth aspect, there is provided a detection system for detecting an anomaly in an oil pressure related operational condition of an internal combustion engine based on comparison with an oil quality monitoring model, the oil quality monitoring model comprising statistical attribute model data previously generated from binned engine model data of the internal combustion engine, in which the binned engine model data has been generated by dividing oil pressure data of engine model data obtained from the internal combustion engine into a predetermined number of engine speed bins, the engine model data comprises oil pressure model data indicative of a oil pressure of the engine and engine speed model data indicative of an engine speed of the engine, the predetermined number of engine speed bins are derived from the engine speed data during a sampling time period, and the binned engine model data is indicative of frequency of occurrence of oil pressures within each bin, the system comprising: a data interface configured to receive, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine; a memory configured to store the engine test data for each test sampling period; and a processor configured to: divide the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model; generate, for each bin of the predetermined number of bins, statistical attribute test data based on the binned engine test data; compare, for each bin, the statistical attribute test data with the statistical attribute model data of the oil quality monitoring model; and generate an alert based on if the comparison between the statistical attribute test data and the statistical attribute model data of the oil quality monitoring model for each test sampling time period meets a predetermined condition.

For example, if the predetermined condition is met, then an alert can be generated indicating that the oil quality or oil pressure may be deviating from optimal conditions, for example, too low an oil pressure, or too high an oil pressure. A user may thus be notified that remedial action might need to be taken, for example, to physically check the oil circulation system, of the quality of the oil. Accordingly, problems related to oil pressure of the engine are more likely to be detected earlier, and possible breakdown may be predicted and pre-emptive action taken. In other words, for example, possible problems with the lubricating oil of the engine may be predicted earlier and before an error fault code is generated by the engine control unit (ECU) of the engine, and remedial actions or repairs may be carried out earlier, this helping to reduce downtime of the vehicle and potentially reducing costs of repair.

Other aspects and features are defined in the appended claims.

### BRIEF DESCRIPTION OF THE DRAWINGS

Examples of the disclosure will now be described by way of example only with reference to the accompanying drawings, in which like references refer to like parts, and in which:

FIG. 1 schematically shows an arrangement for collecting engine data according to examples of the disclosure;

FIG. 2 show example graphs of operational conditions of an engine;

FIG. 3 shows a flowchart of a method for generating an oil quality monitoring model of an internal combustion engine according to examples of the disclosure;

FIG. 4 shows a flow chart of methods for filtering engine model data for use in generating the statistical attribute model according to examples of the present disclosure;

FIG. 5 shows a flow chart of a method for detecting an anomaly in an oil pressure related operational condition of an internal combustion engine according to examples of the disclosure;

FIG. 6 shows a flow chart of methods for filtering engine test data according to examples of the present disclosure;

FIG. 7 shows a graph of an example of comparison of oil pressure with respect to a threshold relationship according to examples of the disclosure;

FIGS. 8a, 8b, and 8c schematically illustrate the application of aggregation logic to generate an alert according to examples of the present disclosure; and

FIG. 9 schematically shows a computer system for implementing methods of examples of the disclosure.

### DETAILED DESCRIPTION

A method and system for generating an oil quality monitoring model, and a method and system for detecting an anomaly in an oil pressure related operational condition is disclosed. In the following description, a number of specific details are presented in order to provide a thorough understanding of the examples of the disclosure. It will be apparent however to a person skilled in the art that these specific details need not be employed in order to practise the examples of the disclosure. Conversely, specific details known to the person skilled in the art are omitted for the purposes of clarity in presenting the examples.

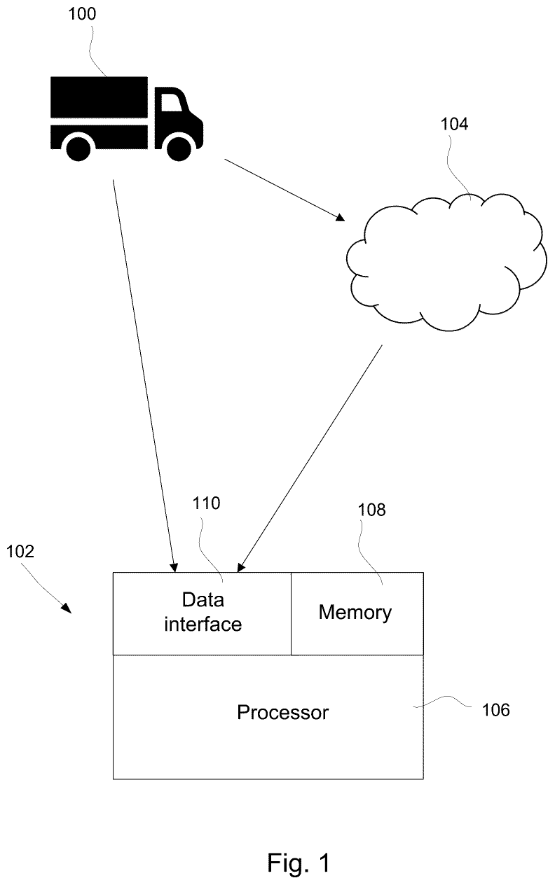

FIG. 1 schematically shows an arrangement for collecting engine data according to examples of the disclosure. A vehicle 100 comprises an internal combustion engine (ICE), such as a common rail diesel engine or a common rail CNG (compressed natural gas) engine. The engine may be provided with sensors, for example mounted on or around the engine, for monitoring operational parameters and conditions of the engine. The sensors may detect different modalities and provide measurements used by an engine management system of the engine to calculate various performance parameters. Such sensors may include as examples, sensors for determining oil pressure of the engine, oil temperature, engine load, and engine speed, although it will be appreciated that other sensors could be used, as appropriate.

Engine data from the sensors may be provided to a telematics gateway unit for communication to a computing system 102, such as a model generation system or detection system as described herein. In some examples, the engine data may be provided to the computing system 102 via a cloud based, or distributed computing network 104. The computing system 102 may be separate from the vehicle 100 and may form part of the distributed computing network 104. Alternatively, the computing system 102 may be provided onboard the vehicle 100. The engine data may include one or more of: oil pressure data indicative of oil pressure of the internal combustion engine; oil temperature data indicative of oil temperature of the internal combustion engine; engine speed data indicative of an engine speed of the engine; and engine load data indicative of an engine load value of the engine.

The computing system 102 may comprise a processor 106, a memory 108, and a data interface 110, which are configured to cooperate together, for example based on computer executable instructions stored in the memory 108 to implement the techniques and methods described herein.

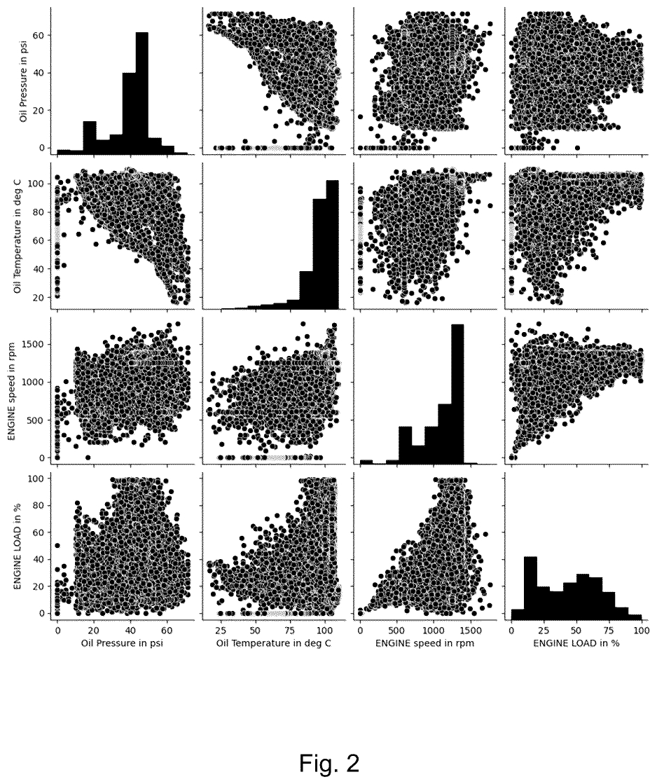

FIG. 2 shows examples of graphs of operational conditions of the internal combustion engine. For example, graphs of oil pressure in psi, oil temperature in degree celsius, engine speed in rpm (revolutions per minute), and engine load in % are shown plotted on the x-axis against engine load in %, engine speed in rpm, oil temperature in degree celsius and oil pressure in psi (pounds per square inch) on the y-axis for each permutation. Engine data obtained from an engine may be used to generate an oil quality monitoring model for an internal combustion engine. Such engine data may also be used to detect an anomaly in an oil pressure related operational condition of an internal combustion engine based on comparison with an oil quality monitoring model. For example, on-board diagnostics parameter IDs (OBD PIDs) according to known standards may be used by the system 102 to obtain the engine data from the vehicle 100.

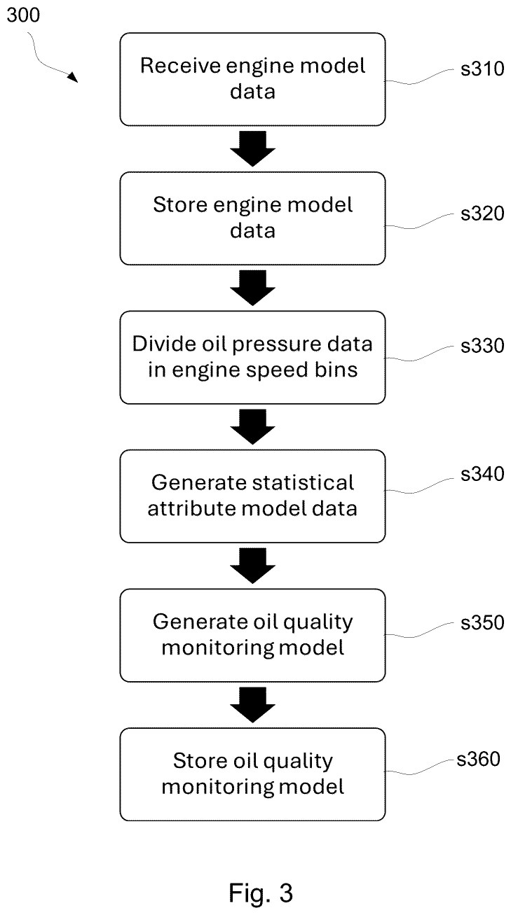

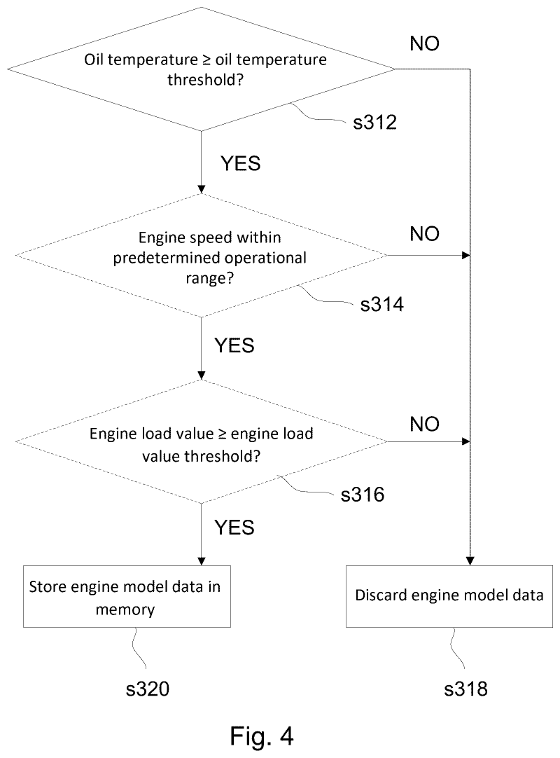

A method 300 for generating an oil quality monitoring model for an internal combustion engine of a vehicle according to examples of the disclosure will now be described with reference to FIGS. 3 and 4. Engine data provided by the vehicle 100 can thus be considered to be engine model data i.e. engine data used for generating the oil quality monitoring model.

At a step s310, the computing system 102 may receive the engine model data from the vehicle 100 via the data interface 110. The engine model data may be obtained periodically during a sequence of sampling time periods during operation of the internal combustion engine for example based on OBD PID communication. For example, each sampling time period may be 1 minute, although other sampling time periods could be used such as every 30 seconds, 90 seconds, 120 seconds. The engine model data may comprise oil pressure data indicative of oil pressure of the internal combustion engine, and engine speed data indicative of an engine speed of the engine, for example as illustrated in FIG. 2.

At a step s320, the engine model data may be stored in the memory 108 for each sampling time period. The sampling time period may be chosen to provide a suitable snapshot of operation of the engine under normal conditions. In some examples, the sampling time period may be chosen such that are at least some fault codes (such as oil pressure related fault codes as well as non-oil pressure associated fault codes) indicated by the engine during the sampling time period. The fault codes may, for example, be used to derive oil quality data as will be described later below.

In some examples, the engine model data may be filtered according to one or more criteria before storing in the memory 108. This may help remove data that may be considered anomalous and so which could degrade the effectiveness of the oil quality monitoring model. The engine model data may also be filtered based on engine load, engine speed, and/or oil temperature, as will be described in more detail later below with reference to FIG. 4. However, it will be appreciated that other operational parameters of the engine could be used to filter the engine model data before storing, as appropriate.

At a step s330, the processor 106 may divide the oil pressure data into a predetermined number of engine speed bins derived from the engine speed data during each sampling time period so as to generate binned engine model data indicative of frequency of occurrence of oil pressures within each bin for each sampling time period. For example, the number of bins when taken together may be chosen to cover all or part of the engine speed values of the engine speed data. In an example, the predetermined number of bins is 4, 5, or 6, although it will be appreciated that any suitable number of bins could be used. A higher number of bins may help provide a more detailed and accurate oil quality monitoring model, but may take longer to generate, and may increase lag time when the oil quality monitoring model is used to detect oil pressure anomalies. A lower number of bins may require less processing to generate, and may help increase detection speed (reduce detection lag), but may be less accurate and so may increase the number of false positives when being used to detect oil pressure anomalies. Therefore, the number of bins may be chosen to provide a balance between detection accuracy and detection lag.

In some examples, the size of each bin is the same, although it will be appreciated they could be different. For example, a first bin (Bin 1) could comprise the engine speed values falling within the first 0-20% of the entire range of engine speed values starting from the lowest value, a second bin (Bin 2) may comprise the engine speed values falling within 20-40% of values within the entire range of engine speed values starting from the lowest value, a third bin (Bin 3) could comprise the engine speed values falling within 40-60% of values within the entire range of engine speed values starting from the lowest value, a fourth bin (Bin 4) could comprise the engine speed values falling within 60-80% of values within the entire range of engine speed values starting from the lowest value, and a fifth bin (Bin 4) could comprise the engine speed values falling within 80-100% of values within the entire range of engine speed values starting from the lowest value.

Instead of the entire range of engine speed values, in some examples, a subset of the entire range of engine speed values may be used instead such as 600-1700 rpm. However, it will be appreciated that other suitable bin divisions and ranges could be used, for example depending on the engine.

In some examples, the oil pressure data may be divided into bins based on engine speed values rather than percentages of the entire engine speed value range, for example Bin 1=600-900 rpm, Bin 2=900-1100 rpm, Bin 3=1100-1300 rpm, Bin 4=1300-1500 rpm, and Bin 5=1500-1700 rpm. The bins may have the same range as each other (e.g. 200 rpm range per bin as in the preceding example), or they may be different from each other. However, it will be appreciated that other techniques and implementations may be used to divide the engine speed data into bins.

At a step s340, the processor 106 may generate, for each bin of the predetermined number of bins and for each sampling time period, statistical attribute model data based on the binned engine model data.

In some examples, the statistical attribute model data may be generated by the processor 106 by fitting a normal Gaussian distribution to the binned engine model data for each bin to determine the mean value of frequency of occurrence of oil pressure and standard deviation of the frequency of occurrence of the oil pressure of the oil pressure data in each bin. The statistical attribute model data may comprise mean value data indicative of the mean value and standard deviation data indicative of the standard deviation for each bin determined by fitting the Gaussian distribution to the binned engine model data.

In other examples, the statistical attribute model data may be generated by the processor 106 by determining, for each bin, one or more percentile ranges of frequency of occurrence of oil pressure of the oil pressure data for the binned engine data, although it will be appreciated that other statistical attributes such as median or mode could be used as well or instead.

At a step s350, the processor 106 may generate the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions. It will be appreciated that the oil quality monitoring model could be generated from statistical model data generated from the fitted Gaussian distributions for each bin, from the percentile ranges for each bin, or a combination of both. It will also be appreciated that other statistical measures could be generated for each bin, and the normal operation model could be generated accordingly based on other appropriate statistical measures.

The operational data of the vehicle may comprise oil quality data. The oil quality data may be derived from one or more fault codes that occurred during a fault code time period that coincides with the sampling time period. The operational data of the vehicle may comprise fuel efficiency data. For example, the fuel efficiency data may correlate with a particular oil quality, with newer oil providing a better fuel efficiency, and the fuel efficiency becoming worse as the oil get older.

In some examples, the oil quality data may be extracted from the fault codes using artificial intelligence and natural language processing such as a large language model, for example to generate a qualitative description of the oil quality. In some examples, the oil quality data may be generated from the fault codes e.g. by applying a large language model to generate a scaled oil quality value, or a binary indicator (e.g. good/bad) for example. For example, based on analysis of the fault codes and/or the fuel efficiency data, the statistical attribute model data may be labelled as being optimal or suboptimal, or otherwise have a quality flag associated with it. For example, if a fault code indicates “oil temperature too high”, “oil pressure too high”, or “oil pressure too low” for a particular sampling time period then the engine model data associated with that sampling time period would be flagged as suboptimal. In other words, for example, if a fault code that is present during a particular sampling time period that is associated with a suboptimal condition of the oil, then the statistical attribute model data for that particular sampling time period may be labelled as suboptimal.

In some examples a sampling time period may be labelled (tagged) as optimal or suboptimal based on user feedback. For example, a user may interact with the computing system 102 via a suitable user interface to input information about oil quality. The oil quality data may comprise the information about oil quality that is input by the user.

For example, a user may input information that the oil was changed e.g. because the oil quality was bad. The date and time at which the oil was changed may be input via the user interface and associated with the label “bad” (suboptimal). Sampling time periods corresponding to a particular duration before the oil was changed may then be tagged (flagged) as being suboptimal. For example, the sampling times periods that occurred within a threshold oil change duration (e.g. 2 weeks) before the oil was changed may be flagged as suboptimal.

In some examples, the oil quality monitoring model may comprise the mean value of frequency of occurrence of oil pressure and standard deviation of the frequency of occurrence of the oil pressure of the oil pressure data for each bin, for example as shown in Table 1 below, where the prefix “M” indicates that the value “Mean” or “Sigma” (standard deviation) is associated with the engine model data:

TABLE 1

| Bin | | Standard |
|---|---|---|
| Number | Mean | Deviation |
| 1 | M-Mean1 | M-Sigma1 |
| 2 | M-Mean2 | M-Sigma2 |
| 3 | M-Mean3 | M-Sigma3 |
| 4 | M-Mean4 | M-Sigma4 |
| 5 | M-Mean5 | M-Sigma5 |

At a step s360, the oil quality monitoring model may stored in the memory 108 for comparison with engine test data accumulated over detection time periods for enabling forecasting of engine oil anomalies of the internal combustion engine based on comparison between the engine test data for each sampling time period and the statistical attribute model data of the oil quality monitoring model.

In some examples, in order to help provide accuracy of the model, the method 300 may be performed for a plurality of internal combustion engines each having the same common attribute so as to generate an oil quality monitoring model for the plurality of internal combustion engines. In other words, engine model data from a plurality of engines having common attributes may be aggregated together in order to generate the oil quality monitoring model.

For example, a common attribute may be that all the engines of the plurality of engines have the same engine capacity (e.g. the same cubic capacity, cc). In other examples, a common attribute may be that all the engines of the plurality of engines have the rated power output (e.g. the same brake horse power, BHP). In some examples, engines of the plurality of engines used to generate the oil quality monitoring model may share more than one common attribute, such as all having the same cubic capacity and rated power output. However, it will be appreciated that other common attributes could be used.

As mentioned above, the engine model data may be filtered according to one or more criteria before storing in the memory 108, and thus excluded from the engine model data being used to generate the oil quality monitoring model. This may help improve the accuracy of the oil quality monitoring model. FIG. 4 shows a flow chart of methods for filtering engine model data for use in generating the statistical attribute model data according to examples of the present disclosure.

The techniques of FIG. 4 may be implemented between the steps s310 and s320 described above as part of the method 300.

At a step s312, the processor may determine if the oil temperature, as indicated by the oil temperature data, is greater than or equal to an oil temperature threshold. If not, then, at the step s318, the associated engine model data may be discarded. For example, oil temperature values that are too low may not provide consistent data for generating the oil quality monitoring model.

If, at the step s312, the oil temperature is determined to be greater than or equal to the oil temperature threshold, then at a step s314, the processor 106 may determine if the engine speed is within a predetermined operational range. If not, then, at the step s318, the associated engine model data may be discarded. For example, particularly low engine or high engine speeds may not provide consistent data for generating the oil quality monitoring model. It will be appreciated that the step s312 could be omitted and engine model data filtering processing could start with step s314.

The engine model data may comprise engine load data indicative of an engine load value of the engine, the engine load data being associated with the engine speed data. If, at the step s314, it is determined that the engine speed is within the predetermined operational range, then, at a step s316, the processor 106 may determine if the engine load value of the engine load data is greater than or equal to a engine load value threshold. If not, then, at the step s318, the associated engine model data may be discarded. For example, low engine load values may often occur when the vehicle is stationary and is idling, or when the vehicle is coasting down a hill, and thus not provide consistent data for generating the oil quality monitoring model.

If, at the step s318, it is determined that the engine load value of the engine load data is greater than or equal to the engine load value threshold, then, at the step s320, the engine model data may be stored in the memory 108 in the same manner as that described above with respect to FIG. 3.

In some examples, the step s314, the step s316, or both may be omitted. It will also be appreciated that order of processing of the steps s312, s314, and s316 could be changed. For example, the step s316 could be carried out before the step s314. If a step is omitted and the criteria of that step is met, then processing proceeds to the next step, which may be storing the engine model data at the step s310 or another filtering step as appropriate.

The steps S312, s314, and s316 can therefore be thought of as filtering steps of a method to help improve the accuracy of the oil quality monitoring model, and thus help reduce the likelihood of false alerts being generated when the oil quality monitoring model is used to detect anomalies in oil pressure related operational condition.

The oil quality monitoring model may be used in detecting an anomaly in an oil pressure related operational condition of an internal combustion engine. The oil quality monitoring model generated as described herein, for example with reference to FIGS. 3 and 4, may be used as a comparison for engine test data acquired during operation of the vehicle, for example during day-to-day operating conditions.

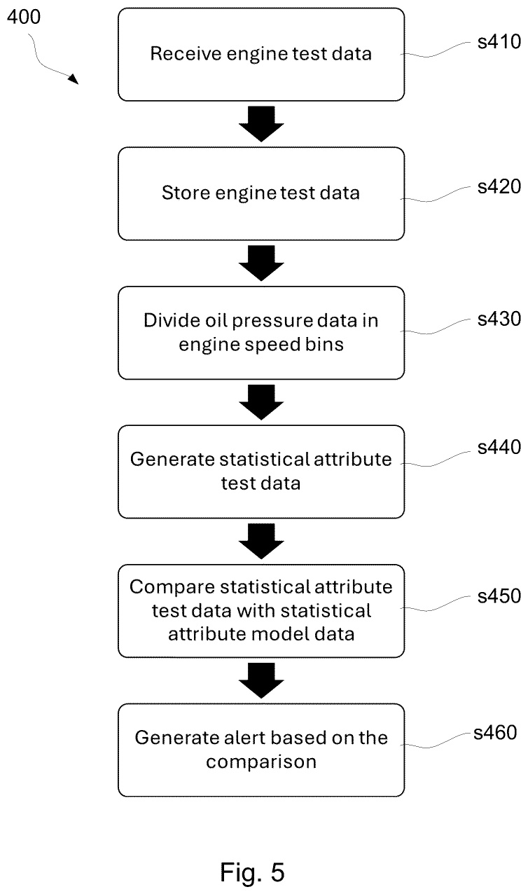

FIG. 5 shows a flow chart of a method for detecting an anomaly in an oil pressure related operational condition of an internal combustion engine.

At a step s410, the computing system 102 may receive engine test data obtained periodically during a sequence of test sampling time periods during operation of the internal combustion engine from an engine management system of the internal combustion engine via the data interface 110. In a similar manner to generating the oil quality monitoring model, the engine test data may comprise oil pressure data indicative of an oil pressure of the internal combustion engine and engine speed data indicative of an engine speed of the engine. The engine test data may be obtained periodically during operation of the ICE, for example based on OBD PID communication. The test sampling time periods may each be the same as each other and may typically be 60 seconds, although other appropriate test sampling time periods such as 30 s, 90 s, 120 s, 180 s could be used.

At a step S420, the engine test data may stored in the memory 108 for each test sampling period. The test sampling period may be chosen to correspond to the sampling time period used for generating the oil quality monitoring model, or it may be different, for example chosen according to lag time to detect an anomaly, since the length of the test sampling period may determine how often anomaly detection can be performed. For example, detection of one or more anomalies may help predict if any engine fault codes might arise, before they actually occur, allowing any necessary remedial action to be taken sooner. Often, if the ECU of an engine generates a fault code, then there may already be a significant problem with the engine. Accordingly, the techniques described herein for detecting anomalies may help address this problem by providing a prediction of such occurrences before they happen, meaning that remediating or pre-emptive actions to prevent significant problems may be able to be taken earlier.

A shorter test sampling period may mean that anomalies in oil pressure may be determined more quickly, but the likelihood of false positives occurring may be increased. A longer test sampling period may help reduce the likelihood of false positives, but increase the length of time between each determination of whether there is an anomaly in the oil pressure of the internal combustion engine.

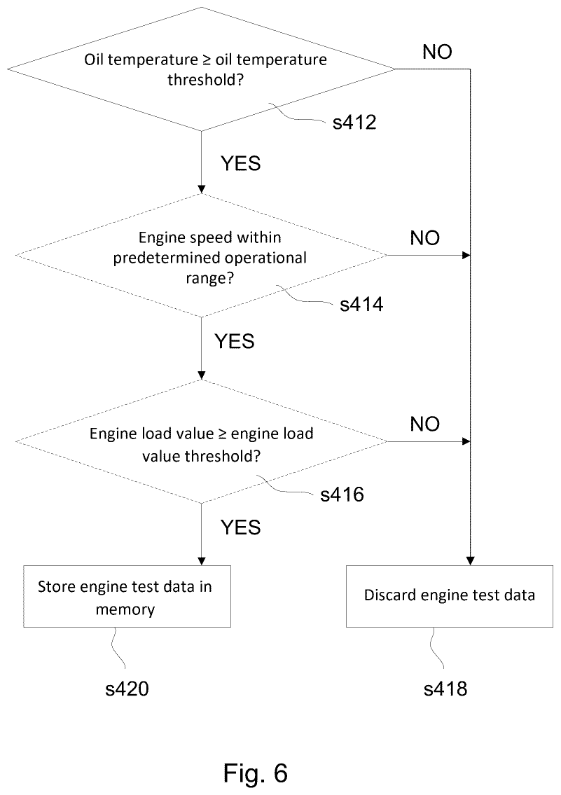

In some examples, the engine test data may be filtered according to one or more criteria before storing in the memory 108. This may help reduce the likelihood of spurious engine test data giving rise to false positives. The engine test data may also be filtered based on oil temperature, engine speed, and/or engine load value, as will be described in more detail later below with reference to FIG. 6. However, it will be appreciated that other operational parameters of the engine could be used to filter the engine test data before storing, as appropriate. In some examples, the same filtering protocols as those used for generating the oil quality monitoring model may be used, although in other examples, different filtering protocols may be used, for example, depending on whether accuracy of detection is being prioritised or speed of detection.

At a step s430, the processor 106 may divide the oil pressure data into a predetermined number of engine speed bins during the test sampling time periods so as to generate binned engine test data indicative of frequency of occurrence of oil pressures within each bin. In examples, the predetermined number of bins corresponds to the same number of bins as those used for the oil quality monitoring model. In examples, the size of the predetermined bins may also be chosen to correspond to those used to generate the oil quality monitoring model. This may help enable comparison of the engine test data with the engine model data via appropriate statistical processing of the engine test data.

At a step s440, the processor 106 may generate, for each bin of the predetermined number of bins, statistical attribute test data based on the binned engine test data.

In some examples, the statistical attribute test data may be generated by the processor 106 by fitting a normal Gaussian distribution to the binned engine test data for each bin to determine the mean value of frequency of occurrence of fuel rail pressure and standard deviation of the frequency of occurrence of the fuel rail pressure of the fuel rail pressure data in each bin. The statistical attribute test data may comprise mean value data indicative of the mean value and standard deviation data indicative of the standard deviation for each bin determined by fitting the Gaussian distribution to the binned engine test data. An example of statistical attribute test data is shown below in table 2 where the prefix “T” indicates that the value “mean” or “Sigma” (standard deviation) is associated with the engine test data:

TABLE 2

| Bin | | Standard |
|---|---|---|
| Number | Mean | Deviation |
| 1 | T-Mean1 | T-Sigma1 |
| 2 | T-Mean2 | T-Sigma2 |
| 3 | T-Mean3 | T-Sigma3 |
| 4 | T-Mean4 | T-Sigma4 |
| 5 | T-Mean5 | T-Sigma5 |

At a step s450, the processor 106 may compare, for each bin, the statistical attribute test data with the statistical attribute model data of the normal operation model.

In some examples, comparing the statistical attribute test data with the statistical attribute model data of the oil quality monitoring model at the step s450 may comprise determining, by the processor 106 for each bin, one or more percentile ranges of frequency of occurrence of oil pressure of the oil pressure data for the binned engine test data, and comparing, by the processor 106 the one or more percentile ranges of frequency of occurrence of oil pressure of the oil pressure data for the binned engine test data with one or more percentile ranges of frequency of occurrence of oil pressure generated from oil pressure data of the binned engine model data.

In other examples, comparing the statistical attribute test data with the statistical attribute model data of the normal operation model at the step s450 may comprise determining, by the processor 106 for each bin, the mean value of frequency of occurrence of oil pressure from the oil pressure data in each bin. The processor 106 may then compare the mean value of frequency of occurrence of the oil pressure for each bin of the statistical attribute test data with the mean value of frequency of occurrence of the oil pressure of the corresponding bin of the statistical attribute data from the oil quality monitoring model. However, other statistical attributes could be compared such as median, mean, and/or standard deviation.

At a step s460, the processor 106 may generate an alert based on if the comparison between the statistical attribute test data and the statistical attribute model data of the oil quality monitoring model for each test sampling time period meets a predetermined condition. For example, if the predetermined condition is met, then an alert may be generated indicating that there is an anomaly in the oil pressure of the internal combustion engine, for example, too low an oil pressure, or too high an oil pressure. A user may thus be notified that remedial action might need to be taken, for example, to physically check the oil circulation system for leaks. Accordingly, problems with the oil pressure and oil circulation system may be more likely to be detected earlier, and possible breakdown may be predicted and pre-emptive action taken.

It will be appreciated that several types of predetermined condition may be implemented depending on the conditions under which it may be desirable to generate an alert. One or more predetermined conditions may also be implemented in such a way as to help reduce the likelihood of false positives being generated (i.e. an alert being generated even if the oil quality is within usual or normal conditions for that engine).

In some examples, the processor 106 may determine for each bin, if an anomaly threshold difference between the mean value of frequency of occurrence of the oil pressure statistical attribute test data for that bin and the mean value of frequency of occurrence oil pressure of the corresponding bin of the statistical attribute model data from the oil quality monitoring model is greater than or equal to a threshold amount, and if so, flagging that bin.

For example, referring to Bin 1 of the statistical attribute test data, the processor 106 may determine whether T-Mean1−M-Mean1≥CThres, where CThres is the threshold amount. If so then Bin 1 would be flagged or otherwise labelled, for example as illustrated in Table 3 below:

TABLE 3

| Bin | | |
|---|---|---|
| Number | | Flag |
| 1 | \|T-Mean1 − M-Mean1\| ≥ CThres? | TRUE |
| 2 | \|T-Mean2 − M-Mean2\| ≥ CThres? | FALSE |
| 3 | \|T-Mean3 − M-Mean3\| ≥ CThres? | TRUE |
| 4 | \|T-Mean4 − M-Mean4\| ≥ CThres? | TRUE |
| 5 | \|T-Mean5 − M-Mean5\| ≥ CThres? | FALSE |

However, it will be appreciated that a bin could be flagged based on the comparison of one or more percentile ranges of frequency of occurrence of oil pressure of the oil pressure data for the binned engine test data with one or more percentile ranges of frequency of occurrence of oil pressure generated from oil pressure data of the binned engine model data. For example, if a percentile range of frequency of occurrence of oil pressure of the oil pressure data for the binned engine test data differs from the corresponding percentile range of frequency of occurrence of oil pressure generated from oil pressure data of the binned engine model data by more than an anomaly threshold amount, then that bin may be flagged.

In an example, the predetermined condition may correspond to the number of flagged bins being greater than or equal to an anomaly threshold number, AnThres. The anomaly threshold number may be selected to be any number between 1 and the total number of bins. A lower anomaly threshold number may mean that the detection method and system is more sensitive and the likelihood of an alert being generated is higher, whereas a higher anomaly threshold number may mean that the detection method and system is less sensitive but reduce the likelihood of false positives.

Referring to Table 3, for example, if the anomaly threshold number is 2, then the predetermined condition would be met, and an alert generated. However, if the anomaly threshold number if 4, then the predetermined condition would not be met, and an alert would not be generated.

While in some of the examples above, the mean values are compared between the statistical attribute model data and the statistical attribute test data, it will be appreciated that other comparisons may be considered. For example, the standard deviation of the statistical attribute test data of each bin may be compared with the corresponding standard deviation of the statistical attribute model data and if the difference is greater than or equal to a standard deviation threshold, then that bin may be flagged in a similar manner to that described above with respect to the mean. If, for example, the number of flagged bins based on the comparison of the respective standard deviations for each bin exceeds a threshold (e.g. a standard deviation anomaly threshold, SDThres), then the predetermined condition may be considered to be met, and an alert generated.

In some implementations, in order to help reduce the likelihood of false positives when generating an alert or alerts, the engine test data may be filtered, for example in a similar manner to that described above with respect to FIG. 4. FIG. 6 shows a flow chart of methods which may be used for filtering engine test data for use in detecting an anomaly in an oil pressure related operational condition of an internal combustion engine. The techniques of FIG. 6 may be implemented between the steps s410 and s420 described above as part of the method 400.

At a step s412, the processor may determine if the oil temperature, as indicated by the oil temperature data, is greater than or equal to an oil temperature threshold. If not, then, at the step s418, the associated engine test data may be discarded. For example, oil temperature values that are too low may not provide consistent data inadvertently raise an alert even if there is no anomaly in the oil pressure related operational condition.

If, at the step s412, the oil temperature is determined to be greater than or equal to the oil temperature threshold, then at a step s414, the processor 106 may determine if the engine speed is within a predetermined operational range. If not, then, at the step s418, the associated engine test data may be discarded. For example, particularly low engine or high engine speeds may not provide consistent data for detecting an anomaly in an oil pressure related operational condition of the engine. It will be appreciated that the step s412 could be omitted and engine model data filtering processing could start with step s414.

The engine test data may comprise engine load data indicative of an engine load value of the engine, the engine load data being associated with the engine speed data. If, at the step s414, it is determined that the engine speed is within the predetermined operational range, then, at a step s416, the processor 106 may determine if the engine load value of the engine load data is greater than or equal to a engine load value threshold. If not, then, at the step s418, the associated engine test data may be discarded. For example, low engine load values may often occur when the vehicle is stationary and is idling, or when the vehicle is coasting down a hill, and thus not provide reliable or consistent data for detecting an anomaly in an oil pressure related operational condition of the engine.

If, at the step s418, it is determined that the engine load value of the engine load data is greater than or equal to the engine load value threshold, then, at the step s420, the engine test data may be stored in the memory 108 in the same manner as that described above with respect to FIG. 5.

In some examples, the step s414, the step s416, or both may be omitted. It will also be appreciated that order of processing of the steps s412, s414, and s416 could be changed. For example, the step s416 could be carried out before the step s414. If a step is omitted and the criteria of that step is met, then processing proceeds to the next step, which may be storing the engine test data at the step s410 or another filtering step as appropriate.

The steps s412, s414, and s416 may therefore be thought of as filtering steps to help reduce the likelihood of false alerts being generated when the oil quality monitoring model is used to detect anomalies in oil pressure related operational conditions of the engine.

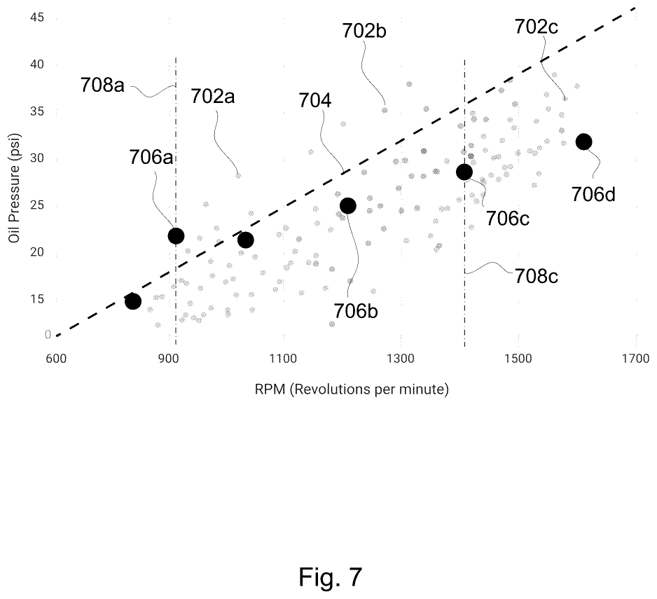

In some examples, comparing, by the processor 106, the statistical attribute test data with the statistical attribute model data of the oil quality monitoring model may comprise fitting by the processor 106, a linear function relating oil pressure of the oil pressure data to at least a portion of engine speed of the engine speed data of the engine model data to define a threshold relationship between the engine speed data and the oil pressure data. This will now be described in more detail with reference to FIG. 7 which shows a graph of an example of comparison of oil pressure with respect to a threshold relationship.

FIG. 7 illustrates a graph of engine speed in rpm (revolutions per minute) on the x-axis with oil pressure in psi (pounds per square inch) on the y-axis. The smaller circles (e.g. 702a, 702b 702c) indicate engine speed and corresponding oil pressure as determined from the engine model data, and may be used to derive a threshold relationship (indicated by the dashed line 704). For example, the threshold relationship may be derived by performing a least squares linear fit to the engine speeds of engine speed data and oil pressures of the oil pressure data from the engine model data, although it will be appreciated that other techniques, such as a piecewise linear fit, could be used to generate the threshold relationship.

Engine test data, comprising engine speed data and oil pressure data for example (e.g. indicated by the larger circles 706a, 706b, 706c, 706d in FIG. 7) may be compared with the threshold relationship by the processor 106 to determine if the predetermined condition is met or not.

For example, the processor 106 may determine if the oil pressure indicated by the engine test data is less than oil pressure defined by the threshold relationship at a particular engine speed associated with that oil pressure indicated by the engine test data. For example, referring to FIG. 7, the oil pressure associated with engine test data 706a is greater than the corresponding oil pressure defined by the threshold relationship 704 at the engine speed indicated by the vertical dashed line 708a, and the oil pressure associated with engine test data 706c is less than the corresponding oil pressure defined by the threshold relationship 704 at the engine speed indicated by the vertical dashed line 708c.

In examples, the predetermined condition may correspond to the engine test data being determined to be less than oil pressure defined by the threshold relationship at a particular engine speed associated with that oil pressure indicated by the engine test data.

For example, referring to FIG. 7, the predetermined condition is met for the engine test data 706c because the oil pressure associated with engine test data 706c is less than the corresponding oil pressure defined by the threshold relationship 704 at the engine speed indicated by the vertical dashed line 708c. In this example, the engine test data 706a does not meet the predetermined condition because the oil pressure associated with engine test data 706a is greater than the corresponding oil pressure defined by the threshold relationship 704 at the engine speed indicated by the vertical dashed line 708a.

In some examples, the engine test data that is compared with the threshold relationship at each engine speed may be aggregated together. For example, the predetermined condition may be met if more than an aggregate threshold of engine test data points that are compared with the threshold relationship have an oil pressure that is less than the corresponding oil pressure for that corresponding engine speed. For example, the aggregate threshold may be a particular percentage of the total number of engine test points that are compared with the threshold relationship (e.g. 50%, 60%, 70%, 80%, although other percentages could be used). In other examples, the aggregate threshold may be a predetermined number of engine test data points, although it will be appreciated that the aggregate threshold could be determined in other suitable manners. This may help reduce the likelihood of false positive alerts being generated.

In some examples, where the predetermined condition is met and an alert is generated, the processor 106 may perform analysis based on the engine test data (such as oil pressure, oil temperature, engine speed and/or engine load) and correlate this with existing diagnostic trouble codes (fault codes). This may be used to predict, based on the oil quality monitoring model, possible causes of the alert being generated such as: low oil level; oil leaks; clogged oil filter; dirty, aerated, or wrong oil; worn engine bearings; faulty oil pressure sensor; oil pump issues; and/or engine overheating, although it will be appreciated that other causes may be predicted as well. For example, information about possible causes may be derived by natural language processing, for example large language model analysis, of the diagnostic trouble codes. For example, if an engine temperature fault code is present, it could be causing low oil pressure. In other words, for example, more generally, possible causes for an alert may be identified based on operational data of the vehicle associated with sampling time periods identified with the one or more suboptimal oil operation conditions.

Large language models (LLMs) such as ChatGPT by OpenAI, Google Gemini, and Microsoft Copilot are known in the art. For example, the system 102 may implement an LLM to analyse DTCs (fault codes) of the vehicle that occurred during each sampling time period to extract the DTCs that are relevant to the condition of the oil quality to generate a list of oil quality related DTCs. The oil quality related DTCs may then be ordered based on the probability that are related to the cause of an alert being generated.

In some examples, a general LLM could be fine-tuned specifically on the fault codes (diagnostic trouble codes, DTCs) and so as to help provide a more content specific output. For example, a suitable prompt may be:
“You are an expert automotive technician tasked with diagnosing and resolving a diagnostic trouble code (DTC). Your goal is to extract the specific component with given cause in order to effectively diagnose and repair the vehicle. Example—
1) cause-to high or too low oil pressure. Extracted components—oil pump, oil tube connectors, oil tubes.
2) cause-oil temperature too high. extracted components—thermostat, thermometer, oil level.
Please provide shortly component for cause—{query}?”

However, it will be appreciated that other suitable prompts could be used.

If there is more than one DTC (fault code) present for a particular sampling time period, then the processor 106 may order the associated DTCs and their causes according to their likelihood. For example, using LLM analysis on the DTCs, the processor 106 may determine components that are in common between the diagnostic trouble codes based on an identification of the components of the vehicle that could be contributing to each fault occurrence. The common components may be those that are identified as being associated with fault occurrences of two of more diagnostic trouble codes. For example if a first DTC, DTCa, is associated with components “oil pump” and “engine thermometer” and a second DTC, DTCb, is associated with components “engine thermometer” and “coolant pump”, then the component “engine thermometer” is a common component between the first DTC, DTCa, and the second DTC, DTCb.

The determination of common components may therefore, for example, be thought of as identifying DTCs associated with more than one component that is found to be in common with the causes associated with the respective DTC codes, for example as identified using LLM analysis.

The processor 106 may order the possible causes based on the identified common components. In examples, the causes may be ordered from most likely to least likely. For example, a cause having the most common components associated with it may be placed as the most likely cause, with a cause having the least components in common for example, may be placed as the least likely.

However, it will be appreciated that other techniques for ordering the causes could be used such as generating a score associated with each cause based on one or more of matching of components between DTCs, user feedback on vehicle symptoms, vehicle repair history, and ordering according to the score, although it will be appreciated that other features could be considered for generating the score.

For example, diagnostic data input to the system 102 may comprise DTCs and their associated causes as determined by LLM analysis may be ordered as shown in Table 4A:

TABLE 4A

| DTCa | DTCb | DTCc |
|---|---|---|
| Cause a1 | Cause b1 | Cause c1 |
| Cause a2 | Cause b2 | Cause c2 |
| Cause a3 | Cause b3 | Cause c3 |
| Cause a4 | Cause b4 | |

In ordering the possible causes based on identified components, the most likely causes may be positioned to appear first in a list of possible causes for each DTC, for example as illustrated in Table 4B (most likely causes are shown in bold type). In other words, for example, the causes associated with the DTCs can be thought of as being ordered so as to place the most likely possible causes first in a list of causes associated with each respective DTC.

TABLE 4B

| DTCa | DTCb | DTCc |
|---|---|---|
| Cause a3 | Cause b4 | Cause c1 |
| Cause a1 | Cause b2 | Cause c2 |
| Cause a2 | Cause b1 | Cause c3 |
| Cause a4 | Cause b3 | |

The possible causes and their ordering may be output by the system 102 on a display via a suitable user interface. A user, such as an engine or repair technician, may then use the information about the possible causes when determining if or how any repairs or pre-emptive action needs to be taken, for example if any parts need replacing or the oil needs changing. This may help provide a more efficient pre-emptive action because the more likely causes could be considered first when conducting a repair or diagnosing a fault with the oil quality.

In other words, more generally, the system 102 may identify possible causes for an alert based on operational data of the vehicle (e.g. DTCs) associated with sampling time periods identified with the one or more suboptimal oil operation conditions.

In some examples, a user such as an engine or repair technician, or vehicle driver, for example may interact with the system 102 via a suitable user interface to input one or more symptoms that may be associated with the engine model data: engine noise due to knocking; reduced engine performance; engine overheating; stalling or rough starts; and/or burning oil smell or excessive exhaust smoke although it will be appreciated that other symptoms may be considered as well. This information may be used to inform the generation of the oil quality monitoring model, for example by associated symptoms that occur with the statistical attribute model data for sampling times periods during which the symptoms occur. In a similar manner, if an alert is generated, an engine technician or vehicle driver, for example may interact with the system 102 via a suitable user interface to input one or more symptoms that may be associated with the engine test data, which may be used to inform about the possible causes.

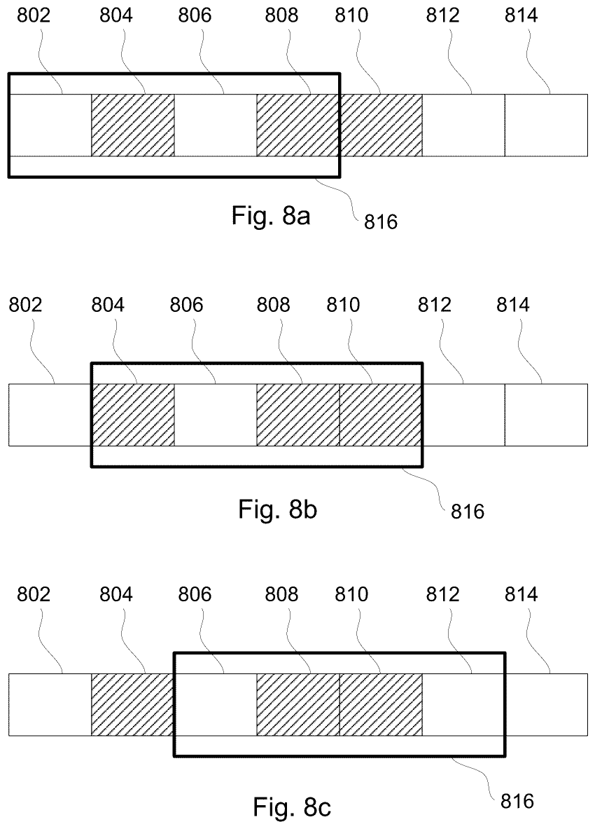

In some examples, it an alert is generated, the system 102 may analyse the statistical attribute model data against which the statistical attribute test data is compared for the test sampling time period associated with the alert to determine if the associated sampling time period for the statistical attribute model data is flagged (tagged) as optimal or suboptimal. In other words, in some examples, the predetermined condition may be associated with if the associated sampling time period for the statistical attribute model data against which the sampling time period is compared is flagged (tagged) as optimal or suboptimal based on the oil quality data. In some examples, in order for an alert to be generated, the predetermined condition based on the comparison between the statistical attribute test data and the statistical attribute model data of the oil quality monitoring model for each test sampling time period may need to be met, as well as the associated sampling time periods of the oil quality monitoring model being labelled (tagged) as suboptimal. FIGS. 8a, 8b, and 8c schematically illustrate the application of aggregation logic to generating an alert according to examples of the present disclosure. As mentioned above, the method 400 may be carried out every test sampling time period sequentially. In other words, for example, once one test sampling time period ends, the method 400 may be implemented again in respect of a next test sampling time period.

FIGS. 8a, 8b, and 8c schematically show a first test sampling time period 802, a second test sampling time period 804, a third test sampling time period 806, a fourth test sampling time period 808, a fifth test sampling time period 810, a sixth test sampling time period 812, and a seventh test sampling time period 814 arranged sequentially. In examples, generating the alert may be based on a windowed time period 816 comprising a sequence of the test sampling time periods, such as the test sampling time periods 802-814. The windowed time period may be controlled by the processor 106 to move over time with respect to the test sampling time periods of the sequence of test sampling time periods.

For example, FIG. 8a shows the windowed time period 816 at a period in time overlapping the test sampling time periods 802-808, FIG. 8b shows the windowed time period 816 at a period in time overlapping the test sampling time periods 804-810, and FIG. 8c shows the windowed time period 816 at a period in time overlapping the test sampling time periods 806-812. Another way of considering this is, for example, that the windowed time period corresponds to a time period coinciding with an integer number of test sampling time periods, and that the test sampling time periods of the sequence of test sampling time periods are considered to move relative to the windowed time period.

In examples, generating the alert comprises turning on the alert if a number of test sampling time periods for which the predetermined condition is met within the windowed time period is greater than or equal to a first threshold number.

In some examples, generating the alert comprises turning off the alert if a number of test sampling time periods for which the predetermined condition is not met within the windowed time period is greater than or equal to a second threshold number.

The first threshold number may be different from the second threshold number, although it will be appreciated that they could be the same as each other.

For example, referring to FIGS. 8a to 8c, test sampling time periods determined as meeting the predetermined condition are shaded with diagonal lines, i.e. test sampling time periods 804, 808, and 810. In an example, the first threshold number may be 3, and the second threshold number may be 2, although it will be appreciated that any suitable numbers could be chosen, for example based on the test sampling time period, the length of the windowed time period, and the desired frequency at which alerts may be generated.

Starting from the position of the windowed time period 816 with respect to the sequence of test sampling time periods in the example of FIG. 8a, the windowed time period comprises two test sampling time periods for which the predetermined condition is met. Therefore, for the example of FIG. 8a, the alert would not be turned on.

In the example of FIGS. 8a-8c, once the windowed time period 816 corresponds to the time period covering the test sampling time periods 804-810 (FIG. 8b), the number of test sampling time periods for which the predetermined condition is satisfied equals the first threshold number, and therefore the alert would be turned on.

When the windowed time period 816 corresponds to the time period covering the test sampling time periods 806-812 (FIG. 8c), the number of test sampling time periods for which the predetermined condition is satisfied equals the second threshold number, and therefore the alert would be turned off. However, if the second threshold number was 3 or more, then in the situation shown in FIG. 8c, the alert would not be turned off until the number of test sampling time periods within the windowed time period which satisfy the predetermined condition becomes greater than or equal to the second threshold number.

The aggregation logic described above with respect to FIGS. 8a-8c may also be applied to the implementation where, in order for alert to be generated, the predetermined condition based on the comparison between the statistical attribute test data and the statistical attribute model data of the oil quality monitoring model for each test sampling time period may need to be met, as well as the associated sampling time periods being labelled (tagged) as suboptimal. In these example implementations, the aggregation logic and windowing may be applied for test sampling periods where both the predetermined condition is met and the test sampling period is labelled (tagged) as being associated with sub-optimal oil quality based on the oil quality monitoring model.

Accordingly, churn between the alert being on and the alert being off may be reduced, thus helping to facilitate better indication of whether there is an issue or not with the oil pressure related operational conditions of the engine, as well as helping reduce data that might need to be sent to a service center based on the alerts.

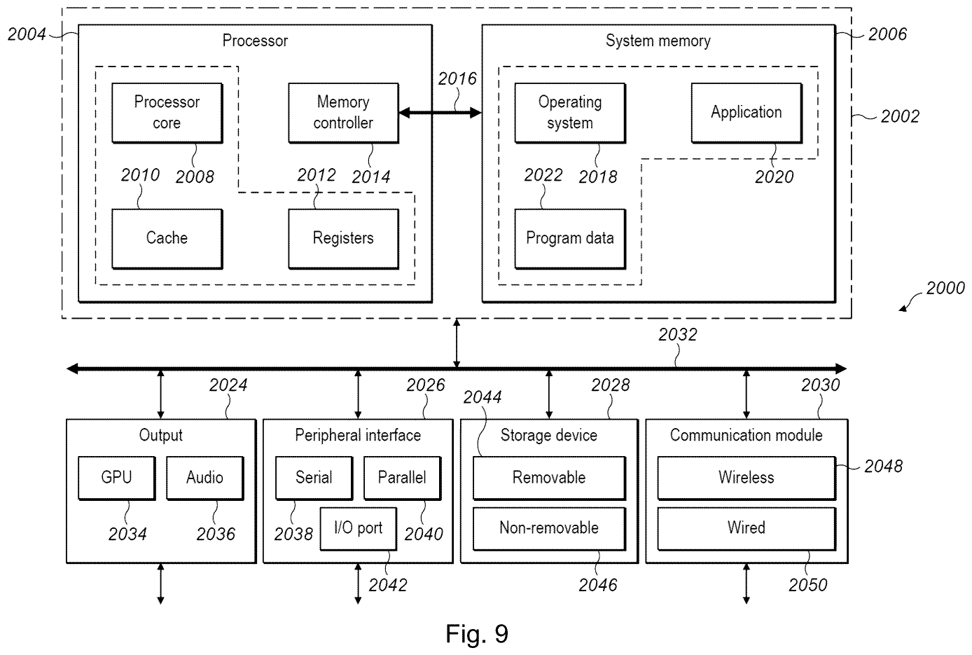

FIG. 9 schematically shows a computer system for implementing methods of examples of the disclosure. In particular, FIG. 9 shows an example of a computing device 2000 for example which may be arranged to implement one or more of the examples of the methods described herein. In examples, the computing device 2000 comprises main unit 2002. The main unit 2002 may comprise a processor 2004 and a system memory 2006. In examples, the processor 2004 may comprise a processor core 2008, a cache 2010, and one or more registers 2012. In examples, the processor core 2008 may comprise one or more processing cores and may comprise a plurality of cores which may run a plurality of threads. The processor 2004 may be of any suitable type such as microcontroller, microprocessor, digital signal processor or a combination of these, although it will be appreciated that other types of processor may be used.

In examples, the processor core 2008 may comprise one or more processing units. In examples, the processor core 2008 comprises one or more of a floating point unit, an arithmetic unit, a digital signal processing unit, or a combination of these and/or plurality of other processing units, although it will be appreciated that other processing units could be used. In examples, the cache 2010 may comprise a plurality of caches such as a level one cache and a level two cache, although other appropriate cache arrangements could be used.

In examples, the processor 2004 comprises a memory controller 2014 operable to allow communication between the processor 2004 and the system memory 2006 via a memory bus 2016. The memory controller 2014 may be implemented as an integral part of the processor 2004, or it may be implemented as separate component.

In examples, the system memory 2006 may be of any suitable type such as non-volatile memory (e.g. flash memory or read only memory), volatile memory (such as random access memory (RAM)), and/or a combination of volatile and non-volatile memory. In examples, the system memory 2006 may be arranged to store code for execution by the processor 2004 and/or data related to the execution. For example, the system memory may store operating system code 2018, application code 2020, and program data 2022. In examples, the application code 2020 may comprise code to implement one or more of the example methods described herein, for example to implement the steps described above with reference to FIGS. 3, 4, 5, and 6. The application code 2020 may be arranged to cooperate with the program data 2022 or other media, for example to provide the functionality described herein.

In examples, the computing device 2000 may have additional features, functionality or interfaces. For example main unit 2002 may cooperate with one or more peripheral devices for example to implement the methods described herein. In examples, the computing device 2000 comprises, as peripheral devices, an output interface 2024, a peripheral interface 2026, a storage device 208, and a communication module 2030. In examples, the computing device comprises an interface bus 2032 arranged to facilitate communication between the main unit 2002 and the peripheral devices.

In examples, the output device 2024 may comprise output devices such as a graphical processing unit (GPU) 2034 and audio output unit 2036 for example arranged to be able to communicate with external devices such as a display, and/or loudspeaker, via one or more suitable ports such as audio/video (A/V) port. In examples, the peripheral interface 2026 may comprise a serial interface 2038, a parallel interface 2040, and a input/output port(s) 2042 which may be operable to cooperate with the main unit 2002 to allow communication with one or more external input and/or output devices via the I/O port 2042. For example, the I/O port 2042 may communication with one or more input devices such as a keyboard, mouse, touch pad, voice input device, scanner, imaging capturing device, video camera, and the like, and/or with one or more output devices such as a 2D printer (e.g. paper printer), or 3D printer, or other suitable output device. For example, engine model data and/or engine test data may be received via the I/O port 2042 and/or the communication module 2030.

In examples, the storage device may comprise removable storage media 2044 and/or non-removable storage media 2046. For example, the removable storage media may be random access memory (RAM), electrically erasable programmable read only memory (EEPROM), read only memory (ROM) flash memory, or other memory technology, optical storage media such as compact disc (CD) digital versatile disc (DVD) or other optical storage media, magnetic storage media such as floppy disc, magnetic tape, or other magnetic storage media. However, it will be appreciated that any suitable type of removable storage media could be used. Non-removable storage media 2046 may comprise a magnetic storage media such as a hard disk drive, or solid state hard drive, or other suitable media, although it will be appreciated that any suitable non-removable storage media could be used. The storage device 2028 may allow access by the main unit 2002 for example to implement the methods described herein.

In examples, the communication module may comprise a wireless communication module 2048 and a wired communication module 2050. For example, the wireless communication module may be arranged to communicate wirelessly via a suitable wireless communication standard for example relating to wifi, Bluetooth, near field communication, optical communication (such as infrared), acoustic communication, or via a suitable mobile telecommunications standard. The wired communication module may allow communication via a wired or optical link for example by Ethernet or optical cable. However, it will be appreciated that any suitable communication module could be used. For example, the communication module may implement the functionality of the data interface 110 described herein.

Referring to FIG. 1, the system 102 may be used to implement the methods 300 or 400 described herein, and the system 102 may for example be implemented by the computing device 2000.

It will be appreciated that in examples of the disclosure, elements of the disclosed methods may be implemented in a computing device (such as the computing device described above with reference to FIG. 9) in any suitable manner. For example, a conventional computing device may be adapted to perform one or more of the methods described herein by programming/adapting one or more processors of the computing device. As such, in examples, the programming/adapting may be implemented in the form of a computer program product comprising computer implementable instructions stored on a data carrier and/or carried by a signal bearing medium, such as floppy disk, hard disk, optical disk, solid state drive, flash memory, programmable read only memory (PROM), random access memory (RAM), or any combination of these or other storage media or signal bearing medium, or transmitted via a network such as a wireless network, Ethernet, the internet, or any other combination of these or other networks.

In other words, in examples, a computer program may comprise computer readable instructions which, when implemented on a computing device, cause the computing device to carry out a method according examples of the disclosure. In examples, a storage medium may comprise the computer program, for example, as mentioned above. It will also be appreciated that other suitable computer architectures could be used such as those based on one or more parallel processors. Furthermore, at least some processing may be implemented on one or more graphical processing units (GPUs). Although computing device 2000 is described as a general purpose computing device, it will be appreciated that this could be implemented in any appropriate device, such as mobile phone, smart phone, camera, video camera, tablet device, server device, one or more distributed computing (e.g. cloud computing) devices with modifications and/or adaptation if appropriate to the features described above, for example dependent on the desired functionality and hardware features.

Further features and examples are defined in the following numbered clauses.

Clause 1. A method for generating an oil quality monitoring model for an internal combustion engine of a vehicle, the method comprising:
receiving, from an engine management system of the internal combustion engine via a data interface, engine model data obtained periodically during a sequence of sampling time periods during operation of the internal combustion engine, the engine model data comprising oil pressure data indicative of oil pressure of the internal combustion engine, and engine speed data indicative of an engine speed of the engine;
storing the engine model data in a memory for each sampling time period;
dividing, by a processor, the oil pressure data into a predetermined number of engine speed bins derived from the engine speed data during each sampling time period so as to generate binned engine model data indicative of frequency of occurrence of oil pressures within each bin for each sampling time period;
generating, by the processor for each bin of the predetermined number of bins and for each sampling time period, statistical attribute model data based on the binned engine model data;
generating, by the processor, the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions; and
storing, in the memory, the oil quality monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

Clause 2. A method according to clause 1, in which:
the engine model data comprises oil temperature data indicative of oil temperature of the internal combustion engine; and
the method comprises determining, by the processor, if the oil temperature is greater than or equal to an oil temperature threshold, and if not, discarding the associated engine model data.

Clause 3. A method according to clause 1 or clause 2, comprising determining, by the processor, if the engine speed of the engine speed data is within a predetermined operational range, and if not, discarding the associated engine model data.

Clause 4. A method according to any preceding clause, in which:
the engine model data comprises engine load data indicative of an engine load value of the engine; and
the method comprises determining, by the processor, if the engine load value of the engine load data is greater than or equal to an engine load value threshold, and if not, discarding the associated engine model data.

Clause 5. A method according to any preceding clause, in which generating the statistical attribute model data comprises fitting a normal Gaussian distribution to the binned engine model data for each bin to determine the mean value of frequency of occurrence of oil pressure and standard deviation of the frequency of occurrence of the oil pressure of the oil pressure data in each bin.

Clause 6. A method according to any preceding clause, in which generating the statistical attribute data comprises determining, for each bin, one or more percentile ranges of frequency of occurrence of oil pressure of the oil pressure data for the binned engine data.

Clause 7. A method according to any preceding clause, comprising performing the method for a plurality of internal combustion engines each having the same common attribute so as to generate an oil quality monitoring model for the plurality of internal combustion engines.

Clause 8. A method according to any preceding clause, in which the operational data of the vehicle comprises oil quality data.

Clause 9. A method according to clause 8, in which oil quality data is derived from one or more fault codes that occurred during a fault code time period that coincides with the sampling time period.

Clause 10. A method according to any preceding clause, in which the operational data of the vehicle comprises fuel efficiency data.

Clause 11. A method for detecting an anomaly in an oil pressure related operational condition of an internal combustion engine based on comparison with an oil quality monitoring model generated according to clause 1, the method comprising:
receiving, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine;
storing the engine test data in a memory for each test sampling period;
dividing, by a processor, the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model;
generating, by the processor for each bin of the predetermined number of bins, statistical attribute test data based on the binned engine test data;
comparing, by the processor for each bin, the statistical attribute test data with the statistical attribute model data of the oil quality monitoring model; and
generating, by the processor, an alert based on if the comparison between the statistical attribute test data and the statistical attribute model data of the oil quality monitoring model for each test sampling time period meets a predetermined condition.

Clause 12. A method according to clause 11, in which:
the engine model data comprises oil temperature data indicative of oil temperature of the internal combustion engine; and
the method comprises determining, by the processor, if the oil temperature is greater than or equal to an oil temperature threshold, and if not, discarding the associated engine model data.

Clause 13. A method according to clause 11 or clause 12, comprising determining, by the processor, if the engine speed data is within a predetermined operational range, and if not, discarding the associated engine test data.

Clause 14. A method according to any of clauses 11 to 13, in which:
the engine test data comprises engine load data indicative of an engine load value of the engine, and
the method comprises determining, by the processor, if the engine load value of the engine load data is greater than or equal to a engine load value threshold, and if not, discarding the associated engine test data.

Clause 15. A method according to any of clauses 11 to 14, in which comparing the statistical attribute test data with the statistical attribute model data of the oil quality monitoring model comprises:
determining, by the processor for each bin, one or more percentile ranges of frequency of occurrence of oil pressure of the oil pressure data for the binned engine test data; and
comparing the one or more percentile ranges of frequency of occurrence of oil pressure of the oil pressure data for the binned engine test data with one or more percentile ranges of frequency of occurrence of oil pressure generated from oil pressure data of the binned engine model data.

Clause 16. A method according to any of clauses 11 to 15, in which comparing the statistical attribute test data with the statistical attribute model data of the oil quality monitoring model comprises:
determining, by the processor for each bin, the mean value of frequency of occurrence of oil pressure from the oil pressure data in each bin; and
comparing the mean value of frequency of occurrence of the oil pressure for each bin of the statistical attribute test data with the mean value of frequency of occurrence of the oil pressure of the corresponding bin of the statistical attribute data from the oil quality monitoring model.

Clause 17. A method according to clause 16, comprising determining, by the processor, for each bin, if an anomaly threshold difference between the mean value of frequency of occurrence of the oil pressure statistical attribute test data for that bin and the mean value of frequency of occurrence oil pressure of the corresponding bin of the statistical attribute model data from the oil quality monitoring model is greater than or equal to a threshold amount, and if so, flagging that bin.

Clause 18. A method according to clause 17, in which the predetermined condition corresponds to the number of flagged bins being greater than or equal to an anomaly threshold number.

Clause 19. A method according to any of clauses 11 to 18, in which:
comparing the statistical attribute test data with the statistical attribute model data of the oil quality monitoring model comprises fitting by the processor, a linear function relating oil pressure of the oil pressure data to at least a portion engine speed of the engine speed data of the engine model data to define a threshold relationship between the engine speed data and the oil pressure data, and:
the method comprises determining, by the processor, if the oil pressure indicated by the engine test data is less than oil pressure defined by the threshold relationship at a particular engine speed associated with that oil pressure indicated by the engine test data; and
the predetermined condition corresponds to the engine test data being determined to be less than oil pressure defined by the threshold relationship at a particular engine speed associated with that oil pressure indicated by the engine test data.

Clause 20. A method according to any of clauses 11 to 19, in which:
the method is carried out every test sampling time period sequentially;
generating the alert is based on a windowed time period comprising a sequence of the test sampling time periods, the windowed time period moving over time with respect to the test sampling time periods of the sequence of detection time periods; and
generating the alert comprises turning on the alert if a number of test sampling time periods for which the predetermined condition is met within the windowed time period is greater than or equal to a first threshold number.

Clause 21. A method according to clause 20, in which:
generating the alert comprises turning off the alert if a number of detection time periods for which the predetermined condition is not met within the windowed time period is greater than or equal to a second threshold number.

Clause 22. A method according to any of clauses 11 to 21, comprising identifying possible causes for an alert based on operational data of the vehicle associated with sampling time periods identified with the one or more suboptimal oil operation conditions.

Clause 23. A method for detecting an anomaly in an oil pressure related operational condition of an internal combustion engine based on comparison with an oil quality monitoring model, the oil quality monitoring model comprising statistical attribute model data previously generated from binned engine model data of the internal combustion engine, in which the binned engine model data has been generated by dividing oil pressure data of engine model data obtained from the internal combustion engine into a predetermined number of engine speed bins, the engine model data comprises oil pressure model data indicative of a oil pressure of the engine and engine speed model data indicative of an engine speed of the engine, the predetermined number of engine speed bins are derived from the engine speed data during a sampling time period, and the binned engine model data is indicative of frequency of occurrence of oil pressures within each bin, the method comprising:
receiving, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine;
storing the engine test data in a memory for each test sampling period;
dividing, by a processor, the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model;
generating, by the processor for each bin of the predetermined number of bins, statistical attribute test data based on the binned engine test data;
comparing, by the processor for each bin, the statistical attribute test data with the statistical attribute model data of the oil quality monitoring model; and
generating, by the processor, an alert based on if the comparison between the statistical attribute test data and the statistical attribute model data of the oil quality monitoring model for each test sampling time period meets a predetermined condition.

Clause 24. A computer program comprising instructions which, when executed by a computer, cause the computer to carry out the method of any of clauses 1 to 10.

Clause 25. A non-transitory tangible computer-readable media having stored thereon a computer program according to clause 24.

Clause 26. A computer program comprising instructions which, when executed by a computer, cause the computer to carry out the method of any of clauses 11 to 22.

Clause 27. A non-transitory tangible computer-readable media having stored thereon a computer program according to clause 26.

Clause 28. A computer program comprising instructions which, when executed by a computer, cause the computer to carry out the method of clause 23.

Clause 29. A non-transitory tangible computer-readable media having stored thereon a computer program according to clause 28.

Clause 30. A model generating system for generating an oil quality monitoring model for an internal combustion engine of a vehicle, the system comprising:
a data interface configured to receive, from an engine management system of the internal combustion engine via a data interface, engine model data obtained periodically during a sequence of sampling time periods during operation of the internal combustion engine, the engine model data comprising oil pressure data indicative of oil pressure of the internal combustion engine, and engine speed data indicative of an engine speed of the engine;
a memory configured to store the engine model data for each sampling time period; and
a processor configured to:
divide the oil pressure data into a predetermined number of engine speed bins derived from the engine speed data during each sampling time period so as to generate binned engine model data indicative of frequency of occurrence of oil pressures within each bin for each sampling time period;
generate, for each bin of the predetermined number of bins and for each sampling time period, statistical attribute model data based on the binned engine model data; and
generate the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions;
in which the memory is configured to store the oil monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

Clause 31. A detection system for detecting an anomaly in an oil pressure related operational condition of an internal combustion engine based on comparison with an oil quality monitoring model generated according to clause 30, the system comprising:
a data interface configured to receive, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine;
a memory configured to store the engine test data for each test sampling period; and
a processor configured to:
divide the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model;
generate, for each bin of the predetermined number of bins, statistical attribute test data based on the binned engine test data;
compare, for each bin, the statistical attribute test data with the statistical attribute model data of the oil quality monitoring model; and
generate an alert based on if the comparison between the statistical attribute test data and the statistical attribute model data of the oil quality monitoring model for each test sampling time period meets a predetermined condition.

Clause 32. A detection system for detecting an anomaly in an oil pressure related operational condition of an internal combustion engine based on comparison with an oil quality monitoring model, the oil quality monitoring model comprising statistical attribute model data previously generated from binned engine model data of the internal combustion engine, in which the binned engine model data has been generated by dividing oil pressure data of engine model data obtained from the internal combustion engine into a predetermined number of engine speed bins, the engine model data comprises oil pressure model data indicative of a oil pressure of the engine and engine speed model data indicative of an engine speed of the engine, the predetermined number of engine speed bins are derived from the engine speed data during a sampling time period, and the binned engine model data is indicative of frequency of occurrence of oil pressures within each bin, the system comprising:
a data interface configured to receive, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine;
a memory configured to store the engine test data for each test sampling period; and
a processor configured to:
divide the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model;
generate, for each bin of the predetermined number of bins, statistical attribute test data based on the binned engine test data;
compare, for each bin, the statistical attribute test data with the statistical attribute model data of the oil quality monitoring model; and
generate an alert based on if the comparison between the statistical attribute test data and the statistical attribute model data of the oil quality monitoring model for each test sampling time period meets a predetermined condition.

Although a variety of techniques and examples of such techniques have been described herein, these are provided by way of example only and many variations and modifications on such examples will be apparent to the skilled person and fall within the spirit and scope of the present invention, which is defined by the appended claims and their equivalents.

1. A method for generating an oil quality monitoring model for an internal combustion engine of a vehicle, the method comprising:
receiving, from an engine management system of the internal combustion engine via a data interface, engine model data obtained periodically during a sequence of sampling time periods during operation of the internal combustion engine, the engine model data comprising oil pressure data indicative of oil pressure of the internal combustion engine, and engine speed data indicative of an engine speed of the engine;
storing the engine model data in a memory for each sampling time period;
dividing, by a processor, the oil pressure data into a predetermined number of engine speed bins derived from the engine speed data during each sampling time period so as to generate binned engine model data indicative of frequency of occurrence of oil pressures within each bin for each sampling time period;
generating, by the processor for each bin of the predetermined number of bins and for each sampling time period, statistical attribute model data based on the binned engine model data;
generating, by the processor, the oil quality monitoring model based on the statistical attribute model data for each bin when taken together for each sampling time period by labelling the statistical attribute model data based on operational data of the vehicle that coincides with the associated sampling time period to identify one or more suboptimal oil operation conditions; and
storing, in the memory, the oil quality monitoring model for comparison with engine test data accumulated over detection time periods for enabling forecasting of engine oil anomalies of the internal combustion engine based on comparison between the engine test data for each sampling time period and the statistical attribute model data of the oil quality monitoring model.

2. The method according to claim 1, wherein:
the engine model data comprises oil temperature data indicative of oil temperature of the internal combustion engine; and
the method comprises determining, by the processor, if the oil temperature is greater than or equal to an oil temperature threshold, and if not, discarding the associated engine model data.

3. The method according to claim 1, further comprising determining, by the processor, if the engine speed of the engine speed data is within a predetermined operational range, and if not, discarding the associated engine model data.

4. The method according to claim 1, wherein:
the engine model data comprises engine load data indicative of an engine load value of the engine; and
the method comprises determining, by the processor, if the engine load value of the engine load data is greater than or equal to an engine load value threshold, and if not, discarding the associated engine model data.

5. The method according to claim 1, wherein the generating the statistical attribute model data comprises fitting a normal Gaussian distribution to the binned engine model data for each bin to determine the mean value of frequency of occurrence of oil pressure and standard deviation of the frequency of occurrence of the oil pressure of the oil pressure data in each bin.

6. The method according to claim 1, wherein the generating the statistical attribute data comprises determining, for each bin, one or more percentile ranges of frequency of occurrence of oil pressure of the oil pressure data for the binned engine data.

7. The method according to claim 1, further comprising performing the method for a plurality of internal combustion engines each having the same common attribute so as to generate an oil quality monitoring model for the plurality of internal combustion engines.

8. (canceled)

9. The method according to claim 1, wherein oil quality data is derived from one or more fault codes that occurred during a fault code time period that coincides with the sampling time period.

10. (canceled)

11. A method for detecting an anomaly in an oil pressure related operational condition of an internal combustion engine based on comparison with an oil quality monitoring model generated according to claim 1, the method comprising:
receiving, from an engine management system of the internal combustion engine via a data interface, engine test data obtained periodically during a sequence of test sampling time periods during operation of the internal combustion engine, the engine test data comprising oil pressure data indicative of an oil pressure of the internal combustion engine and engine speed data indicative of an engine speed of the engine;
storing the engine test data in a memory for each test sampling period;
dividing, by a processor, the oil pressure data into a predetermined number of engine speed bins during the test sampling time periods so as to generate binned engine test data indicative of frequency of occurrence of oil pressures within each bin, in which the predetermined number of bins corresponds to the same number of bins as those used for the oil quality monitoring model;
generating, by the processor for each bin of the predetermined number of bins, statistical attribute test data based on the binned engine test data;
comparing, by the processor for each bin, the statistical attribute test data with the statistical attribute model data of the oil quality monitoring model; and
generating, by the processor, an alert based on if the comparison between the statistical attribute test data and the statistical attribute model data of the oil quality monitoring model for each test sampling time period meets a predetermined condition.

12. The method according to claim 11, wherein:
the engine model data comprises oil temperature data indicative of oil temperature of the internal combustion engine; and
the method comprises determining, by the processor, if the oil temperature is greater than or equal to an oil temperature threshold, and if not, discarding the associated engine model data.

13. The method according to claim 11, further comprising determining, by the processor, if the engine speed data is within a predetermined operational range, and if not, discarding the associated engine test data.

14. The method according to claim 11, wherein:
the engine test data comprises engine load data indicative of an engine load value of the engine, and
the method comprises determining, by the processor, if the engine load value of the engine load data is greater than or equal to a engine load value threshold, and if not, discarding the associated engine test data.

15. The method according to claim 11, wherein the comparing the statistical attribute test data with the statistical attribute model data of the oil quality monitoring model comprises:
determining, by the processor for each bin, one or more percentile ranges of frequency of occurrence of oil pressure of the oil pressure data for the binned engine test data; and
comparing the one or more percentile ranges of frequency of occurrence of oil pressure of the oil pressure data for the binned engine test data with one or more percentile ranges of frequency of occurrence of oil pressure generated from oil pressure data of the binned engine model data.

16. The method according to claim 11, wherein the comparing the statistical attribute test data with the statistical attribute model data of the oil quality monitoring model comprises:
determining, by the processor for each bin, the mean value of frequency of occurrence of oil pressure from the oil pressure data in each bin; and
comparing the mean value of frequency of occurrence of the oil pressure for each bin of the statistical attribute test data with the mean value of frequency of occurrence of the oil pressure of the corresponding bin of the statistical attribute data from the oil quality monitoring model.

17. The method according to claim 16, further comprising determining, by the processor, for each bin, if an anomaly threshold difference between the mean value of frequency of occurrence of the oil pressure statistical attribute test data for that bin and the mean value of frequency of occurrence oil pressure of the corresponding bin of the statistical attribute model data from the oil quality monitoring model is greater than or equal to a threshold amount, and if so, flagging that bin.

18. The method according to claim 17, wherein the predetermined condition corresponds to the number of flagged bins being greater than or equal to an anomaly threshold number.

19. The method according to claim 11, wherein:
the comparing the statistical attribute test data with the statistical attribute model data of the oil quality monitoring model comprises fitting by the processor, a linear function relating oil pressure of the oil pressure data to at least a portion engine speed of the engine speed data of the engine model data to define a threshold relationship between the engine speed data and the oil pressure data, and:
the method comprises determining, by the processor, if the oil pressure indicated by the engine test data is less than oil pressure defined by the threshold relationship at a particular engine speed associated with that oil pressure indicated by the engine test data; and
the predetermined condition corresponds to the engine test data being determined to be less than oil pressure defined by the threshold relationship at a particular engine speed associated with that oil pressure indicated by the engine test data.

20. The method according to claim 11, wherein:
the method is carried out every test sampling time period sequentially;
generating the alert is based on a windowed time period comprising a sequence of the test sampling time periods, the windowed time period moving over time with respect to the test sampling time periods of the sequence of detection time periods; and
generating the alert comprises turning on the alert if a number of test sampling time periods for which the predetermined condition is met within the windowed time period is greater than or equal to a first threshold number.

21. The method according to claim 20, wherein:
generating the alert comprises turning off the alert if a number of detection time periods for which the predetermined condition is not met within the windowed time period is greater than or equal to a second threshold number.

22. The method according to claim 11, further comprising identifying possible causes for an alert based on operational data of the vehicle associated with sampling time periods identified with the one or more suboptimal oil operation conditions.

23-32. (canceled)
